(19) United States
(12) Patent Application Publication
(10) Pub. No.: US 2019/0240437 A1
(43) Pub. Date: Aug. 8, 2019

(54) External Fluid Communication Apparatus

(71) Applicant: Roger James Baker, Aurora, CO (US)

(72) Inventor: Roger James Baker, Aurora, CO (US)

(21) Appl. No.: 16/267,389

(22) Filed: Feb. 5, 2019

Related U.S. Application Data
(60) Provisional application No. 62/626,693, filed on Feb. 6, 2018.

(51) Int. Cl. A61M 16/06

(52) U.S. Cl. CPC A61M 16/0666; A61M 15/08; A61M 16/0683

(57) ABSTRACT

An external fluid communication apparatus that is placed adjacent to a surface, the fluid communication apparatus including a flexible surrounding sidewall having a first end portion and an opposing second end portion with a longitudinal axis therebetween, wherein the first end portion is adapted to connect to a fluid source containing a fluid and the second end portion is adapted to feed a consumer of the fluid with the sidewall operationally communicating the fluid from the first end portion to the second end portion, the sidewall further having an inner surface and an opposing outer surface. Further included in the external fluid communication apparatus is a flexible textured matte skin element disposed on the outer surface that is operational to not reflect ambient light.

### RELATED APPLICATION

This application claims the benefit of U.S. provisional patent application Ser. No. 62/626,693 filed on Feb. 6, 2018 by Roger James Baker of Aurora, Colo., U.S.

### TECHNICAL FIELD

The present invention generally relates to a portable fluid communication apparatus used typically to feed a fluid from a source to a consumer of the fluid. More particularly, the present invention is a portable fluid communication apparatus that feeds a gas from a gas source to the consumer of the gas that is typically a person needing respiratory assistance wherein the portable fluid communication apparatus interfaces with a nasal cavity of the person to feed the gas to lungs of the person.

### BACKGROUND OF INVENTION

There are many forms of respiratory assistance scenarios that include lung disease, airway constriction, heart disorders, neuromuscular disease, and so on, wherein the respiratory assistance is typically in the form of oxygen concentration of the air inhaled by the individual therethrough their nasal cavity, so that there is not breathing help per se, just an increase in oxygen concentration of the air inhaled through the individual's nasal cavity, wherein the portable communication apparatus is termed a small bore nasal cannula which is structurally in the form of a clear plastic tube that loops over the ear lobe being placed across the cheek and terminating in a partial insertion disposed within a nostril of the individual.

The nasal cannula is typically worn by the individual continuously and as it is on the individuals face, it is always highly visible thus this not being a desirable situation as it would be difficult to somewhat hide the nasal cannula under clothing with it being prominently centered on the face, thus a desirable solution would be to make the nasal cannula less visually perceptible in relation to the facial skin surface while retaining its intended function.

In the nasal cannula prior art in U.S. Pat. No. 9,180,270 to Kapust, et al., disclosed is a non-invasive nasal ventilation system, the system comprising: at least one outer tube with a proximal lateral end of the at least one outer tube adapted to extend to a side of a nose, wherein the at least one outer tube includes a wall and a throat section; at least one coupler at a distal section of the at least one outer tube for impinging at least one nostril and positioning the at least one outer tube relative to the at least one nostril. Also in Kapust, at least one jet nozzle is positioned within the at least one outer tube at the proximal lateral end and in fluid communication with a pressurized gas supply; at least one pressure tap disposed within the at least one outer tube in fluid communication with a transducer; at least one opening in the distal section adapted to be in fluid communication with the at least one nostril. Further in Kapust, at least one aperture is defined through the wall of the at least one outer tube in fluid communication with ambient air, wherein the at least one aperture is in downstream proximity to the at least one pressure tap, also the system can include flesh-toned tubing and parts, there can be “skins” for the system—personalize or individual system covers.

Further in the nasal cannula prior art in U.S. Pat. No. 6,701,924 to Land Jr., et al. disclosed is a nasal filter that is a breathing aid that would filter pollutants and contaminants from the air, thus improving the quality of the air inhaled by individuals who suffer respiratory ailments and allergies to air borne pollutants. Land Jr. is designed to fit just inside the nostrils, the nasal filter would consist of a spring clip having flanged two-pronged ends with an oval shaped filtering member on each end. The filtering elements in Land Jr. would be made of a medium grade cotton rolled into an appropriate configuration to fit the nostril, and could possibly be impregnated with oxymetazoline to aid in the treatment of nasal congestion. The flanged ends in Land Jr., would secure the device in place during exhalation, and could also be useful in replacing the disposable filtering elements. Further in Land Jr., the spring clip could be offered in clear or flesh-toned plastic and could also be offered in a variety of sizes to fit different user's noses.

Continuing in the nasal cannula prior art in U.S. Pat. No. 6,004,342 to Filis disclosed is an insert to be placed in nasal passages comprising a pair of connected elastomeric members wherein the exterior surface of each member is molded to conform to a nasal passage so that the exterior surface substantially abuts the interior surface of the nasal passage, wherein the elastomeric members are connected by a connecting strip, and only the connecting strip remains outside of the nasal passages. Filis works to improve air flow therethrough the nasal cavities to help reduce snoring. Further in Filis the elastomeric members are flesh toned in color so as to be less visible when inserted into the nasal passages.

Next, in the nasal cannula prior art in U.S. Pat. No. 8,235,051 to Soderberg, disclosed is an internal nose-dilating device, comprising two conical inserts, which each have four walls and two openings: a small opening, which during use is oriented inwards towards the nasal cavity, and a large opening, which is oriented out from the nose, the inserts being connected by means of a connecting member, which during use is the only part that remains on the outside of the nose. In Soderberg, the four walls of the inserts are convex at their central portions and provided with a substantially horizontal recess, the recess being arranged along the four walls of the inserts and extending at least partly along the circumference on the outside thereof to form a bulge in the four walls of the inserts; in that the inserts have at least four apertures, the four apertures are disposed substantially perpendicular to the horizontal recess; and that a reinforced portion is provided along a rounded edge of the small opening, thereby allowing the inserts to be turned inside out. Preferably in Soderberg, the material is transparent or flesh-colored to ensure that the inserts are visibly discrete during use.

Yet, further in the nasal cannula prior art in U.S. Pat. No. 8,833,369 to Dolezal, disclosed is a nasal air filtering device comprising: a first tubular body comprising an anterior end and a posterior end, and an inner wall of the first tubular body defining a first passageway therethrough, and further comprising a first rim disposed circumferentially about the first tubular body and extending radially outwardly therefrom. Also in Dolezal, a second tubular body comprising an anterior end and a posterior end is included, and an inner wall of the second tubular body defining a second passageway therethrough, plus further comprising a second rim disposed circumferentially about the second tubular body and extending radially outwardly therefrom; a first filtering medium extending from the inner wall of the first tubular body across the first passageway and a second filtering medium extending from the inner wall of the second tubular body across the second passageway.

Further, Dolezal has a connecting member coupled to the first tubular body and the second tubular body; wherein the anterior end and posterior end of the first tubular body each define a plane and the anterior end and posterior end of the second tubular body each define a plane, wherein the first filtering medium includes at least one generally flat surface that defines a plane which is nonparallel to the planes defined by the anterior end and posterior end of the first tubular body, and the second filtering medium includes at least one generally flat surface that defines a plane which is nonparallel to the planes defined by the anterior end and posterior end of the second tubular body. The Dolezal nasal air filtering device has at least a portion of the nasal filtering device that comprises a flesh toned material.

Thus the above references give an idea of the current state of the art in the nasal cannula arts wherein most the references are teaching air flow management in nasal passages being in Filis and Soderberg, filtering in Land and Dolezal, and flow management in Kapust.

There remains a need for a more complete system for making the nasal cannula less visible upon the face of the individual wearing the nasal cannula to reduce the apprehension of the individual wearing the nasal cannula in public while at the same time retaining the nasal cannula function and not creating any added burden for the individual wearer of the nasal cannula.

### SUMMARY OF INVENTION

Broadly, the present invention is of an external fluid communication apparatus placed adjacent to a surface, the fluid communication apparatus including a flexible surrounding sidewall having a first end portion and an opposing second end portion with a longitudinal axis therebetween, wherein the first end portion is adapted to connect to a fluid source containing a fluid and the second end portion is adapted to feed a consumer of the fluid with the sidewall operationally communicating the fluid from the first end portion to the second end portion, the sidewall further having an inner surface and an opposing outer surface. Further included in the external fluid communication apparatus is a flexible textured matte skin element disposed on the outer surface that is operational to not reflect ambient light.

These and other objects of the present invention will become more readily appreciated and understood from a consideration of the following detailed description of the exemplary embodiments of the present invention when taken together with the accompanying drawings, in which;

### BRIEF DESCRIPTION OF DRAWINGS

FIG. 1 shows an perspective view of the external fluid communication apparatus that is being used in its normal operating position being placed on the individual consumer of the fluid with the external fluid communication apparatus adjacent to the surface of the individuals face;

FIG. 2 shows a side elevation view of the external fluid communication apparatus that is being used in its normal operating position being placed on the individual consumer of the fluid with the external fluid communication apparatus adjacent to the surface of the individuals face;

FIG. 3 shows cross section 3-3 from FIG. 1 of the external fluid communication apparatus that shows the flexible surrounding sidewall, the inner and outer surfaces, the flexible textured matte skin element, and the dimension of the surrounding sidewall all resting on the surface;

FIG. 4 shows cross section 4-4 from FIG. 2 of the external fluid communication apparatus that shows the flexible surrounding sidewall, the inner and outer surfaces, the flexible textured matte skin element, and the dimension of the surrounding sidewall with the perpendicular position shown of the dimension to the longitudinal axis all resting on the surface; and

FIG. 5 shows view 5-5 from FIG. 2 of the external fluid communication apparatus that shows the flexible surrounding sidewall, the inner and outer surfaces, and the flexible textured matte skin element, all resting on the surface.

### REFERENCE NUMBERS IN DRAWINGS

50 External fluid communication apparatus, nasal cannula
55 Surface, patient's face
60 Ambient light
65 Individual consumer
70 Flexible surrounding sidewall
75 First end portion of the sidewall 70
80 Second end portion of the sidewall 70
85 Longitudinal axis of the sidewall 70
90 Fluid source
95 Fluid
100 Adaption of the second end portion 80 to feed the consumer 65 with the fluid 95
105 Operational communication of the fluid 95 from the first end portion 75 to the second end portion 80
110 Inner surface of the surrounding sidewall 70
115 Outer surface of the surrounding sidewall 70
120 Flexible textured matte skin element
125 Protrusions of the textured matte skin element 120
130 Valleys of the textured matte skin element 120
135 Elevation distance between the protrusions 125 and the valleys 130
140 Dimension of the surrounding sidewall 70
145 Perpendicular position of the surrounding sidewall 70 dimension 140 to the longitudinal axis 85
150 Multisided surface
155 Different flesh tone shades
160 Visually perceptible minimal differential relation from the multisided surface 150 or the external fluid communication apparatus 50 relative to the surface 55

### DETAILED DESCRIPTION

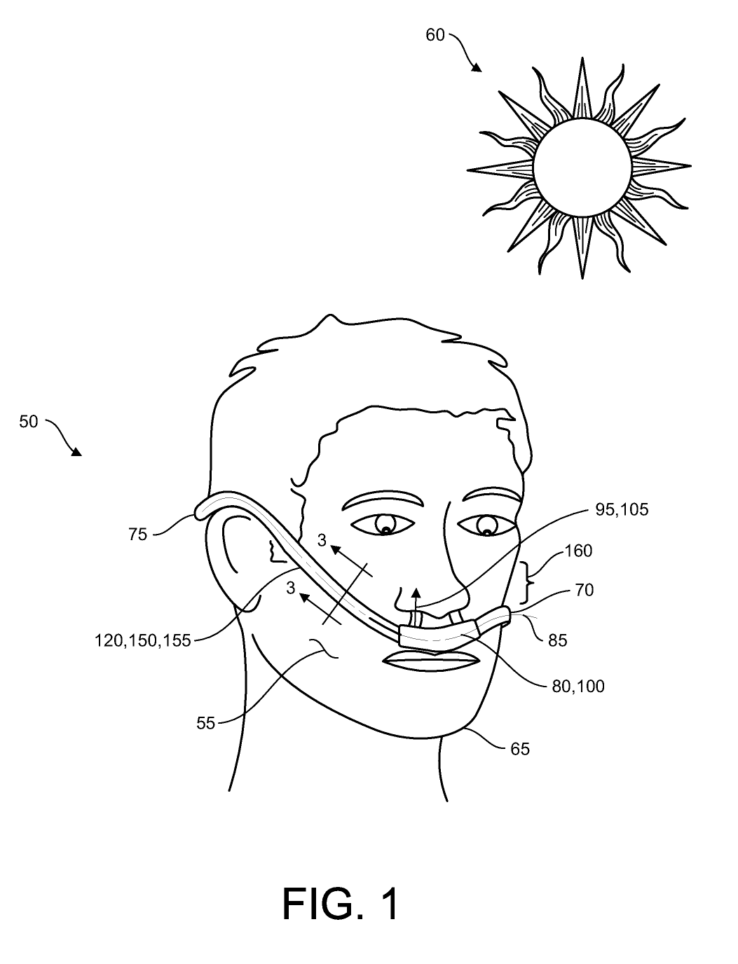

Starting with FIG. 1 shown is a perspective view of the external fluid communication apparatus 50 that is being used in its normal operating position being placed on the individual consumer 65 of the fluid 95 with the external fluid communication apparatus 50 adjacent to the surface 55 of the individuals 65 face.

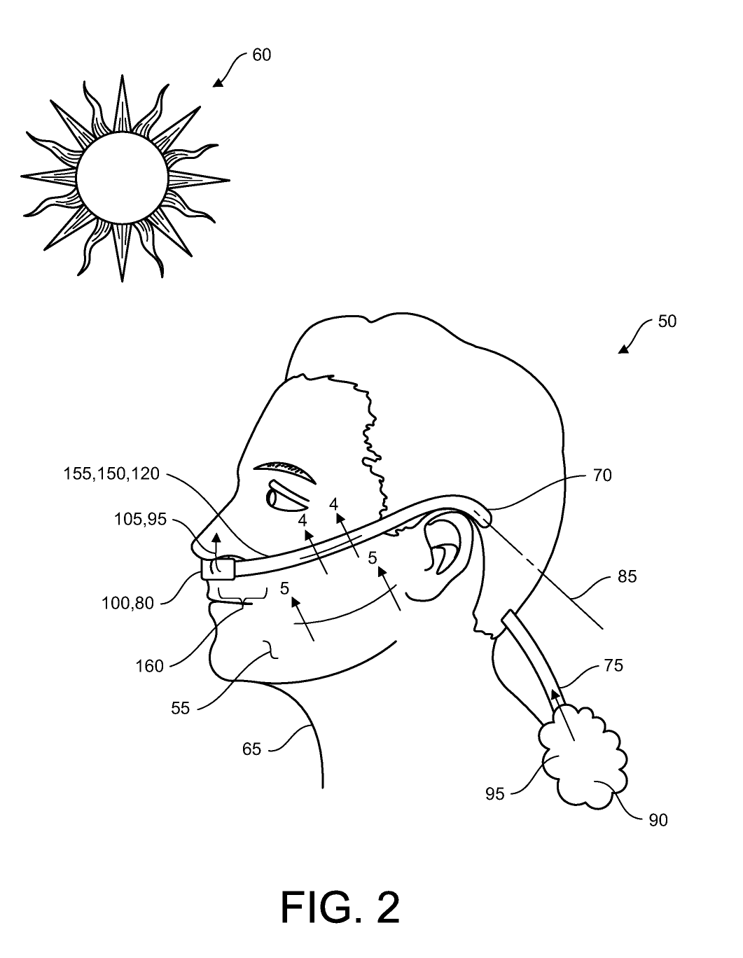

Next, FIG. 2 shows a side elevation view of the external fluid communication apparatus 50 that is being used in its normal operating position being placed on the individual consumer 65 of the fluid 95 with the external fluid communication apparatus 50 adjacent to the surface 55 of the individuals face.

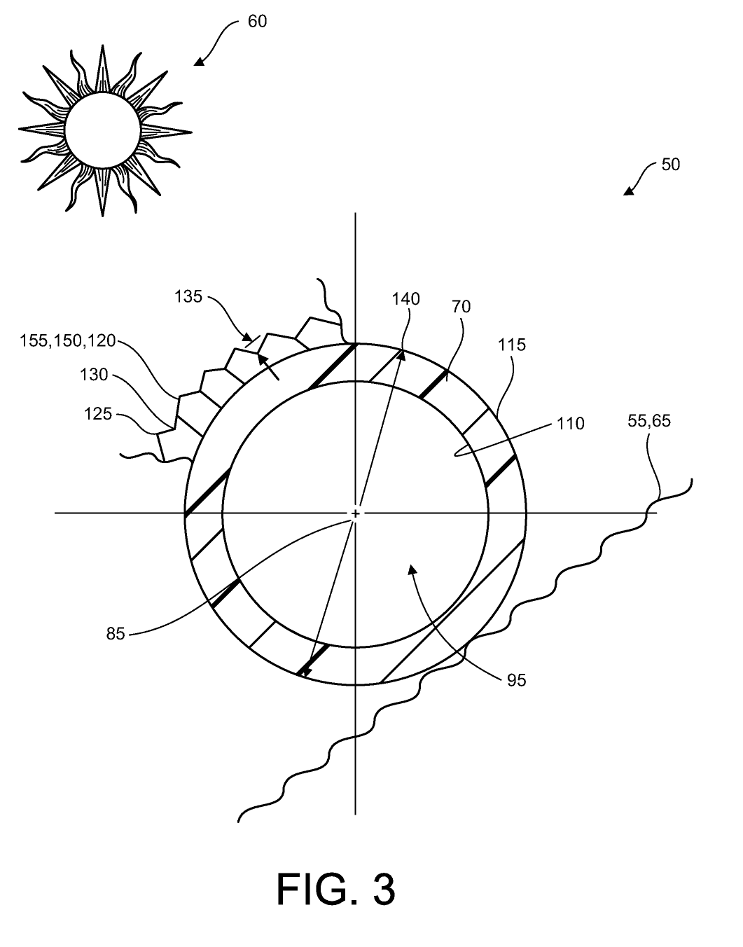

Continuing, FIG. 3 shows cross section 3-3 from FIG. 1 of the external fluid communication apparatus 50 that shows the flexible surrounding sidewall 70, the inner 110 and outer 115 surfaces, the flexible textured matte skin element 120, and the dimension 140 of the surrounding sidewall 70 all resting on the surface 55.

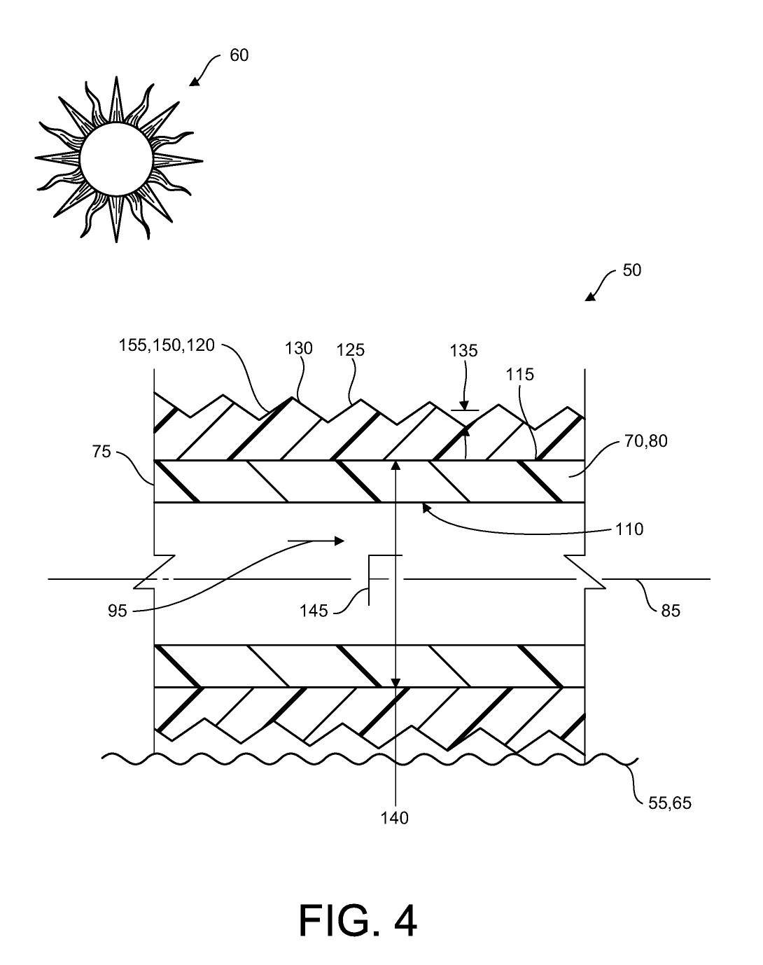

Further, FIG. 4 shows cross section 4-4 from FIG. 2 of the external fluid communication apparatus 50 that shows the flexible surrounding sidewall 70, the inner 110 and outer 115 surfaces, the flexible textured matte skin element 120, and the dimension 140 of the surrounding sidewall 70 with the perpendicular position 145 shown of the dimension 140 to the longitudinal axis 85 all resting on the surface 55.

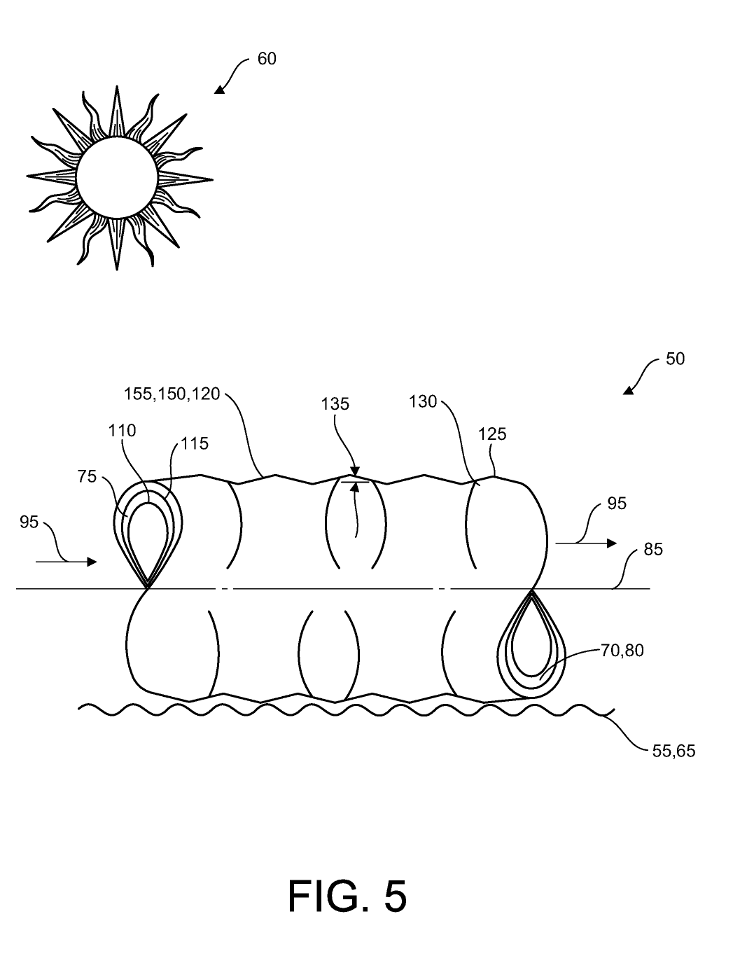

Moving onward, FIG. 5 shows view 5-5 from FIG. 2 of the external fluid communication apparatus 50 that shows the flexible surrounding sidewall 70, the inner 110 and outer 115 surfaces, and the flexible textured matte skin element 120, all resting on the surface 55.

Broadly, the present invention is of the external fluid communication apparatus 50 placed adjacent to the surface 55, the fluid communication apparatus 50 including the flexible surrounding sidewall 70 having the first end portion 75 and the opposing second end portion 80 with the longitudinal axis 85 therebetween, see FIGS. 3 and 4 in particular and FIG. 5. Wherein the first end portion 75 is adapted to connect to the fluid source 90 containing the fluid 95 and the second end portion 80 is adapted 100 to feed the consumer 65 of the fluid 95 with the sidewall 70 operationally communicating 105 the fluid 95 from the first end portion 75 to the second end portion 80, the sidewall 70 further having the inner surface 110 and the opposing outer surface 115, also see in particular FIGS. 1 and 2, plus FIGS. 3, 4, and 5. Further included in the external fluid communication apparatus 50 is the flexible textured matte skin element 120 disposed on the outer surface 115 that is operational to reduce ambient light 60 reflection, as best shown in FIGS. 3 and 4, plus FIG. 5.

As an option for the external fluid communication apparatus 50, wherein the flexible textured matte skin 120 has the plurality of protrusions 125 and the adjacent valleys 130 that define the elevation distance 135 from the protrusion 125 to the valley 130, wherein the elevation distance 135 is less than ten percent (10%) of the surrounding sidewall 70 dimension 140 that is perpendicular 145 to the longitudinal axis 85 to operationally give the multisided surface 150 a minimally visually perceptible 160 image relative to the surface, see FIGS. 3 and 4 in particular, plus also see FIG. 5.

Another option for the external fluid communication apparatus 50, wherein the flexible textured matte skin 120 is a plurality of different flesh tone shades 155 to operationally minimize visual perception 160 of the fluid communication apparatus 50 relative to the surface 55, as best shown in FIG. 5, further FIGS. 3 and 4, and also FIGS. 1 and 2.

### CONCLUSION

Accordingly, the present invention of an external fluid communication apparatus has been described with some degree of particularity directed to the embodiments of the present invention. It should be appreciated, though; that the present invention is defined by the following claims construed in light of the prior art so modifications of the changes may be made to the exemplary embodiments of the present invention without departing from the inventive concepts contained therein.

1. An external fluid communication apparatus placed adjacent to a surface, said fluid communication apparatus comprising:
(a) a flexible surrounding sidewall having a first end portion and an opposing second end portion with a longitudinal axis therebetween, wherein said first end portion is adapted to connect to a fluid source containing a fluid and said second end portion is adapted to feed a consumer of the fluid with said sidewall operationally communicating the fluid from said first end portion to said second end portion, said sidewall further having an inner surface and an opposing outer surface; and
(b) a flexible textured matte skin element disposed on said outer surface that is operational to reduce ambient light reflection.

2. An external fluid communication apparatus according to claim 1 wherein said flexible textured matte skin has a plurality of protrusions and adjacent valleys that define an elevation distance from said protrusion to said valley, wherein said elevation distance is less than ten percent (10%) of a surrounding sidewall dimension that is perpendicular to said longitudinal axis to operationally give a multisided surface that is minimally visually perceptible relative to the surface.

3. An external fluid communication apparatus according to claim 2 wherein said flexible textured matte skin is a plurality of different flesh tone shades to operationally minimize visual perception of said fluid communication apparatus relative to the surface.

4. A nasal cannula placed adjacent to a patients face, said nasal cannula comprising:
(a) a flexible surrounding sidewall having a first end portion and an opposing second end portion with a longitudinal axis therebetween, wherein said first end portion is adapted to connect to a fluid source containing a fluid and said second end portion is adapted to supply the fluid to the patient, with said sidewall operationally communicating the fluid from said first end portion to said second end portion, said sidewall further having an inner surface and an opposing outer surface; and
(b) a flexible textured matte skin element disposed on said outer surface that is operational to reduce ambient light reflection.

5. A nasal cannula according to claim 4 wherein said flexible textured matte skin has a plurality of protrusions and adjacent valleys that define an elevation distance from said protrusion to said valley, wherein said elevation distance is less than ten percent (10%) of a surrounding sidewall dimension that is perpendicular to said longitudinal axis to operationally give a multisided surface that is minimally visually perceptible relative to the patient's face.

6. A nasal cannula according to claim 5 wherein said flexible textured matte skin is a plurality of different flesh tone shades to operationally minimize visual perception of said fluid communication apparatus relative to the patient's face.
